(19) United States
(12) Patent Application Publication
(10) Pub. No.: US 2020/0073627 A1
(43) Pub. Date: Mar. 5, 2020

(54) METHOD OF STREAMING MOTION PICTURE

(71) Applicant: Kaoru YAMAZAKI, TOKYO (JP)

(72) Inventor: Kaoru YAMAZAKI, TOKYO (JP)

(21) Appl. No.: 16/676,714

(22) Filed: Nov. 7, 2019

Related U.S. Application Data
(63) Continuation of application No. PCT/JP2018/017748, filed on May 8, 2018.

(30) Foreign Application Priority Data
May 8, 2017 (JP) 2017-092654

(51) Int. Cl. G06F 3/16; A61B 5/024; A61B 7/04

(52) U.S. Cl. CPC G06F 3/165; H04N 21/4223; A61B 7/04; A61B 5/02444

(57) ABSTRACT

A method of streaming a motion picture, comprises: transmitting a motion picture data containing an image signal specifying an image and a heart rate signal specifying a heart rate of a person appearing in the image; and reproducing a heart sound based on the heart rate signal in synchronization with the image reproduced with the image signal. A viewer of a motion picture is allowed to have identification with the character in the motion picture by hearing or listening to the heart sound. The viewer of the motion picture possibly enjoys more a realistic feeling through the motion picture than ever.

### CROSS-REFERENCE TO RELATED APPLICATION

This application is a continuing application, filed under 35 U.S.C. § 111(a), of International Application PCT/JP2018/017748, filed May 8, 2018, which is based upon and claims the benefit of priority from the prior Japanese Patent Application No. 2017-92654 filed on May 8, 2017, the contents of which are incorporated herein by reference.

### FIELD

The embodiments discussed herein is related to a method of streaming a motion picture.

### BACKGROUND

JP Application Publication No. 2013-207489 discloses a motion picture streaming system. A video signal is decoded in a decoding section. A decoded video signal is then supplied to a displaying monitor. The video is displayed on the displaying monitor. An audio signal is reproduced into sound waves through loudspeakers. The sound waves reach the ears of a user.

### SUMMARY

JP Application Publication No. 2015-514443 discloses a special effect apparatus allowing viewers to experience a realistic feeling not only with video and audio but also through the senses other than the sense of sight and the sense of hearing in a movie theater. The special effect apparatus serves to cause the viewers of a movie to have identification with the character in the movie. The viewers of the movie thus enjoy the realistic feeling. However, it requires a tremendous amount of cost for establishment of equipment. And it cannot be realized in a facility other than a movie theater.

It is accordingly an object in one aspect of the present invention to provide a method of streaming a motion picture capable of causing a viewer to have a realistic feeling with a simplified equipment.

According to a first aspect of the invention, a method of streaming a motion picture, comprises: transmitting a motion picture data containing an image signal specifying an image and a heart rate signal specifying a heart rate of a person appearing in the image; and reproducing a heart sound based on the heart rate signal in synchronization with the image reproduced with the image signal.

The human heart rate and the change of the heart rate reflect the human emotion. A person senses the emotion of the others by hearing or listening to the heart sound. A viewer of a motion picture is allowed to have identification with the character in the motion picture by hearing or listening to the heart sound. The viewer of the motion picture possibly enjoys more a realistic feeling through the motion picture than ever.

According to a second aspect of the invention, there is provided a heart sound reproduction apparatus: a receiving section configured to receive a heart rate signal specifying a pace of a heart rate; a storage section configured to hold a heart sound data specifying a sound wave of a heart sound; and a reproduction section configured to reproduce the heart sound based on the heart sound data in accordance with the pace specified in the heart rate signal.

The heart rate signal is required to specify the pace of the heart rate. The heart rate signal is not required to specify the heart sound. It is not necessary to patch a microphone onto the chest to pick up the heart sound. An available common device such as a pulse sensor may be used to generate the heart rate signal. The heart sound can be reproduced in accordance with the heart rate in a simplified manner.

According to a third aspect of the invention, there is provided a method of reproducing a heart sound, comprising: receiving a heart rate signal specifying a pace of a heart rate; gaining from a storage section a heart sound data specifying a sound wave of a heart sound; and reproducing the heart sound based on the heart sound data in accordance with the pace specified in the heart rate signal.

The heart rate signal is required to specify the pace of the heart rate. The heart rate signal is not required to specify the heart sound. It is not necessary to patch a microphone onto the chest to pick up the heart sound. An available common device such as a pulse sensor may be used to generate the heart rate signal. The heart sound can be reproduced in accordance with the heart rate in a simplified manner.

According to a fourth aspect of the invention, there is provided an image capturing apparatus comprising: an imaging device configured to generate an image signal; a microphone configured to generate an audio signal; a pulse sensor configured to generate a heart rate signal specifying at least a pace of a heart rate; and a mixing device configured to output the audio signal mixed with the heart rate signal.

A motion picture is made based on the combination of the image specified in the image signal and the audio sound specified in the audio signal. Since the audio signal is mixed with the heart rate signal to create a motion picture, the heart rate signal is incorporated into a general format of a conventional motion picture. A peculiar format is not necessary to transmit the heart rate signal. The heart rate signal can be incorporated in a facilitated manner.

According to a fifth aspect of the invention, there is provided a heartbeat reproduction system comprising: gaining a heart rate signal from an individual one of pulse sensors; transmitting to a designated one of reproduction terminals the heart rate signal associated with the designated one of the reproduction terminals; and causing the designated one of the reproduction terminals to reproduce a heart sound based on the heart rate signal received at the designated one of the reproduction terminals.

The pulse sensors are respectively attached to individuals. A heart rate signal is transmitted to the reproduction terminals related to the corresponding one of the individuals. A heart sound is reproduced for the designated one of the reproduction terminals based on the heart rate signal assigned to the designated one of the reproduction terminals. A particular one of the individuals is allocated to the individual one of the reproduction terminals. The heart sound of the particular one of the individuals is reproduced for the designated one of the reproduction terminals.

According to a sixth aspect of the invention, there is provided a pulse sensor apparatus comprising: a bandage having a contact section configured to contact an instep and wound around the instep and an arch of a foot; and a pulse sensor supported on the contact section and configured to detect a pulse from an arteria dorsalis pedis.

The pulse sensor apparatus is attached to the foot in front of the ankle for detecting the pulse of a person. The pulse sensor apparatus does not hinder the movement of the foot around the ankle. Moreover, the human body is relatively rigid at the arch of the foot, so that the pulse sensor apparatus is sufficiently prevented from displacement. A wearer may feel a comfortable fitness. The pulse sensor apparatus may operate to detect the pulse from the arteria dorsalis pedis with an unconscious wearing of the pulse sensor apparatus.

According to a seventh aspect of the invention, there is provided a method of reproducing a heart sound, comprising: gaining a device identifier of a headphone when the headphone is connected to a body of a reproduction terminal; comparing the device identifier gained with a list of registered identifier or identifiers in the reproduction terminal so as to check whether or not the device identifier gained is registered in the reproduction terminal; and outputting an audio signal of a heart sound to the headphone if the registration of the device identifier is confirmed.

Unless the registration of the headphone is confirmed, the audio signal is not outputted to the headphone. The audio signal is supplied solely to a particular headphone. A headphone capable of reproducing the heart sound may be distinguished from other headphones.

According to an eighth aspect of the invention, there is provided a heartbeat reproduction apparatus comprising: a receiving section configured to receive a heart rate signal specifying a pace of a heart rate; a storage section configured to store a heartbeat data specifying a heartbeat; and a vibrating source configured to reproduce the heartbeat based on the heartbeat data in accordance with the pace specified in the heart rate signal.

The heart rate signal is only required to specify the pace of the heart rate. The heart rate signal is not required to specify the heart sound. It is not necessary to patch a microphone onto the chest to pick up the heart sound. An available common device such as a pulse sensor may be used to generate the heart rate signal. The heartbeat (the vibration of a heartbeat) may be reproduced at the pace of the heart rate in a simplified manner.

According to a ninth aspect of the invention, there is provided a heartbeat reproduction apparatus comprising: a receiving section configured to receive a heart rate signal specifying a heart rate generated in a sensor; and a vibrating source configured to vibrate in accordance with the heart rate specified in the heart rate signal.

The human heart rate and the change of the heart rate reflect the human emotion. The human being senses the emotion of the others by feeling the heartbeat. The wearer of the heartbeat reproduction apparatus is allowed to enjoy identification with the wearer of the sensor. The human being may be released from loneliness by feeling the heartbeat.

According to a tenth aspect of the invention, there is provided a heartbeat reproduction apparatus comprising: a receiving section configured to receive a heart rate signal specifying a heart rate generated in a sensor; and a sound source configured to generate a sound in accordance with the heart rate specified in the heart rate signal.

The human heart rate and the change of the heart rate reflect the human emotion. The human being senses the emotion of the others by hearing or listening to the heart sound. The wearer of the heartbeat reproduction apparatus is allowed to enjoy identification with the wearer of the sensor. The human being may be released from loneliness by hearing or listening to the heart sound.

As described above, the disclosed apparatus enables to provide a method of streaming a motion picture capable of causing the viewer of the motion picture to enjoy a realistic feeling in a facilitated manner.

The object and advantages of the embodiment will be realized and attained by means of the elements and combinations particularly pointed out in the appended claims. It is to be understood that both the foregoing general description and the following detailed description are exemplary and explanatory and are not restrictive of the embodiment, as claimed.

### BRIEF DESCRIPTION OF THE DRAWINGS

FIG. 1 is a schematic view illustrating the structure of a motion picture streaming system according to a first embodiment;

FIG. 2 is a schematic view illustrating the structure of an emotion distributing system according to a second embodiment;

FIG. 3 is a schematic view illustrating the structure of a pulse sensor apparatus according to a specific example;

FIG. 4A and FIG. 4B are schematic views illustrating specific examples of vibrators;

FIG. 5 is a schematic view illustrating the structure of a heartbeat reproduction apparatus according to a third embodiment;

FIG. 6 is a schematic view illustrating the structure of a heartbeat reproduction apparatus according to a fourth embodiment;

FIG. 7 is a schematic view illustrating the structure of a heartbeat reproduction apparatus according to a fifth embodiment;

FIG. 8 is a schematic view illustrating the structure of a heartbeat reproduction apparatus according to a sixth embodiment; and

FIG. 9 is a schematic view illustrating the structure of a heartbeat reproduction apparatus according to a seventh embodiment.

### DESCRIPTION OF EMBODIMENTS

Embodiments of the present invention will be explained below with reference to the accompanying drawings.

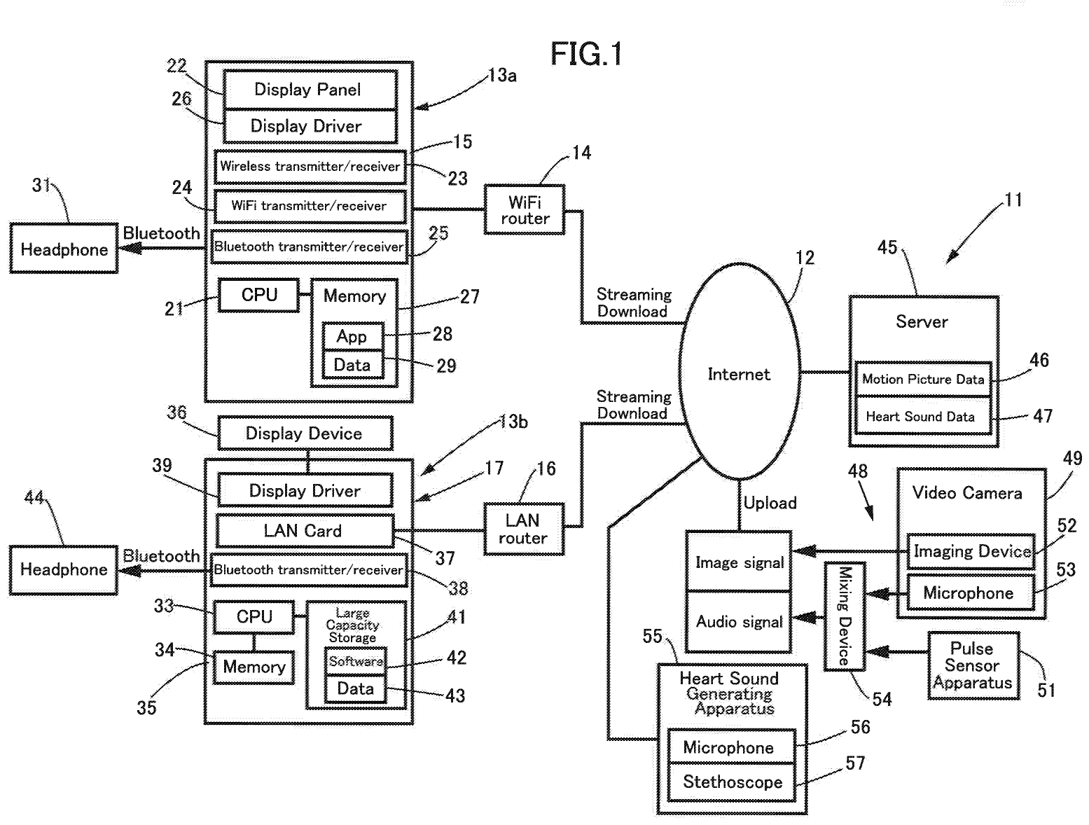

FIG. 1 schematically illustrates the structure of a motion picture streaming system 11 according to a first embodiment. The motion picture streaming system 11 includes reproduction terminals (heart sound reproduction apparatus) 13a, 13b arbitrarily connectable to the Internet 12. The reproduction terminal 13a according to a first specific example comprises a smartphone 15 connected to the Internet 12 through a WiFi router 14. The reproduction terminal 13b according to a second specific example comprises a personal computer 17 connected to the Internet 12 through a LAN router 16. The smartphone 15 may be connected to the Internet 12 through a mobile phone network. The personal computer 17 may be connected to the Internet 12 through the WiFi router 14.

The smartphone 15 includes a CPU (central processing unit) 21 incorporated in a main enclosure, and a display panel 22 embedded in the front surface of the main enclosure. The display panel 22 defines a rectangular screen, for example, on the surface. The main enclosure encloses a wireless transmitter/receiver circuit 23 connected to a base station of a mobile phone network, a WiFi transmitter/receiver circuit 24 connected to the WiFi router 14, a Bluetooth transmitter/receiver circuit 25, a display driver 26 configured to control the display on the screen, and other electronic circuits, all connected to the CPU 21.

The CPU 21 is configured to receive a motion picture data from the Internet 12 through the WiFi transmitter/receiver circuit 24 (or the wireless transmitter/receiver circuit 23). The motion picture data contains an image data specifying an image, and an audio signal specifying the audio sound related to the image. A heart rate signal is mixed in the audio signal so as to specify the heart rate of the character in the image. Here, the heart rate signal is designed to specify the pace of the heart rate. The pace of the heartbeat may be expressed based on the intervals of the pulse signals, other rhythmic signal, or the like, for example. The pace of the heartbeat may be defined by the intervals between the first heart sounds or the intervals between the second heart sounds, for example.

A memory (storage section) 27 is connected to the CPU 21. The memory 27 is configured to keep a mobile application program (smartphone app) 28 and a heart sound data 29. The heart sound data 29 is designed to specify the sound waves of heart sounds. Since sound may be reproduced with the vibration of a diaphragm, the heart sound data 29 may also function as a heartbeat data specifying heartbeat. The CPU 21 is configured to execute the mobile application program 28. The execution of the mobile application program 28 enables the CPU 21 to receive motion picture data, to gain the heart sound data 29 from the memory 27, and to reproduce the heart sound based on the heart sound data 29 in accordance with the pace specified in the heart rate signal.

A headphone 31 is connected to the smartphone 15. A cable may be used to connect the headphone 31 to the smartphone 15, or any wireless means is used to connect the headphone 31 to the smartphone 15. Here, Bluetooth (a registered trademark) is used to connect the headphone 31 to the smartphone 15. An audio signal is supplied to the headphone 31 from the CPU 21 through the Bluetooth transmitter/receiver circuit 25. The loudspeaker of the headphone 31 emits the sound waves in the air in response to the action of the diaphragm, for example. The headphone 31 is worn on the head of a person. The headphone 31 may preferably be a bone conduction headphone.

The CPU 21 is configured to reproduce the image based on the image signal with the assistance of an image processing unit and to reproduce the sound based on the audio signal. The display driver 26 serves to display the image on the screen of the display panel 22. The CPU 21 is configured to decode the heart rate signal from the audio signal. The CPU 21 (a reproduction section) is configured to reproduce the heart sound based on the heart rate signal in synchronization with the image reproduced with the image signal. The ears of a person receive from the headphone 31 the sound related to the image as well as the heart sound of the character in synchronization with the image.

The personal computer 17 includes: a main enclosure 35 enclosing a CPU (central processing unit) 33 and a memory 34; and a display device 36 connected to the main enclosure 35 and having a display panel defining a rectangular screen, for example. The main enclosure 35 and the display device 36 may be separate from each other like a desktop personal computer, or may be coupled with each other through hinges like a notebook personal computer. Alternatively, the personal computer 17 may be a tablet personal computer having a display panel within a main enclosure. The main enclosure 35 encloses a LAN card 37, a Bluetooth transmitter/receiver circuit 38, a display driver 39 configured to control the display on the screen, and other electronic circuits, all connected to the CPU 33. The CPU 33 is configured to receive a motion picture data from the Internet 12 through the LAN card 37.

A large capacity storage device (storage section) 41 is connected to the CPU 33. The large capacity storage device 41 is configured to keep an application software program, hereinafter “software program”, 42 and a heart sound data 43. The heart sound data 43 specifies the sound waves of heart sounds. The heart sound data 43 may also function as a heartbeat data specifying heartbeat. The execution of the software program 42 enables the CPU 33 (a receiving section) to receive motion picture data, to gain the heart sound data 29 from the large capacity storage device 41, and to reproduce the heart sound based on the heart sound data 29 in accordance with the pace specified in the heart rate signal.

A headphone 44 is connected to the personal computer 17. A cable may be used to connect the headphone 44 to the personal computer 17, or any wireless means is used to connect the headphone 44 to the personal computer 17. Here, Bluetooth is used to connect the headphone 44 to the personal computer 17. The headphone 44 may have the structure similar to the aforementioned headphone 31.

The CPU 33 is configured to reproduce the image based on the image signal with the assistance of an image processing unit and to reproduce the sound based on the audio signal. The display driver 39 serves to display the image on the screen of the display device 36. The CPU 33 is configured to decode the heart rate signal from the audio signal. The CPU 33 (a reproduction section) is configured to reproduce the heart sound based on the heart rate signal in synchronization with the image reproduced with the image signal. The ears of a person receive from the headphone 44 the sound related to the image as well as the heart sound of the character in synchronization with the image.

The motion picture streaming system 11 includes a server 45 connected to the Internet 12. The server 45 is configured to hold motion picture data 46 and heart sound data 47. The execution of the mobile application program 28 at the smartphone 15 enables establishment of a communication path between the smartphone 15 and the server 45. The mobile application program 28 serves to provide a user interface to the application executed at the server 45.

The motion picture streaming system 11 includes an image capturing apparatus 48 connected to the Internet 12. The image capturing apparatus 48 has: a video camera 49 configured to generate an image; and a pulse sensor apparatus (a pulse sensor) 51 configured to detect the pulse (namely, the heart rate) of a wearer who wears the pulse sensor apparatus 51. The pulse sensor apparatus 51 may detect the pulse from any of a superficial temporal artery, a facial artery, a common carotid artery, an axillary artery, a brachial artery, a radial artery, an ulnar artery, a femoral artery, a popliteal artery, a posterior tibial artery, and an arteria dorsalis pedis. Since the pulse sensor apparatus 51 is attached to a person appearing in the image, the pulse can be detected in synchronization with the heart rate of the person in the image. The pulse sensor apparatus generates a heart rate signal specifying at least the pace of the heartbeat. The pace of the heartbeat may be expressed based on the intervals of the pulse signals, other rhythmic signal, or the like, for example, in the aforementioned manner.

The video camera 49 includes: an imaging device 52 configured to generate an image signal; and a microphone 53 configured to generate an audio signal. The image signal is designed to specify an image projected on the imaging device 52 through a lens or lenses, for example. The audio signal is designed to specify the voice of a person or persons as well as the environmental sound picked up in connection with the image projected on the imaging device 52. A mixing processing circuit (a mixing device) 54 is connected to at least the microphone 53 and the pulse sensor apparatus 51. The mixing processing circuit 54 is configured to output the audio signal containing the heart rate signal as sound mixed with the audio sound. The audio signal may include a pulse signal specifying the first sounds or the second sounds of the heartbeat, for example. Here, the motion picture data is generated in accordance with a conventional format including the image signal and the audio signal.

The image capturing apparatus 48 is connected to the server 45 via the Internet, for example. The motion picture can be uploaded to the server 45. The server 45 is configured to keep motion picture data 46. The motion picture data 46 may be grouped for sets of images.

The motion picture streaming system 11 includes a heart sound generating apparatus 55 connected to the Internet 12. The heart sound generating apparatus 55 includes a microphone 56 configured to gain the sound waves of the heartbeat from the human body. A stethoscope 57 is connected to the microphone 56. The stethoscope 57 is configured to detect the heart sound from any of the aortic valve area, the pulmonary valve area, the tricuspid valve area, the mitral valve area and the Erb's point and transmit the amplified heart sound to the microphone 56. The microphone 56 is configured to output an electric signal specifying the sound waves of the heartbeat, namely a heart sound data. The pace of the heartbeat may arbitrarily be set. The heart sound data may be prepared for various paces. The heart sound data may be uploaded to the server 45. The server 45 is configured to keep the heart sound data 47. The heart sound data 47 may be grouped for individual persons.

Next, explanation will be made on the action of the motion picture streaming system 11. A user of the smartphone 15, the viewer, is allowed to tap the screen to start the mobile application program 28, for example. The mobile application program 28 takes the heart sound data 29 in. The heart sound data 29 may be downloaded from the server 45 upon the installation of the mobile application program 28 and stored in the memory 27 of the smartphone 15, or may be downloaded from the server 45 upon reproduction of the heart sound and stored in the memory 27 of the smartphone 15.

In the case of a so-called video streaming, where the motion picture data 46 is kept on the server, when the viewer selects a motion picture file based on the manipulation on the screen of the smartphone 15, the server 45 transmits to the Internet 12 the motion picture data containing an image data specifying an image, and a heart rate signal specifying the heart rate of a character appearing in the image. The motion picture data is supplied to the CPU 21 of the smartphone 15 through the Internet 12 along the time axis. The CPU 21 separates the heart rate signal from the audio signal in the motion picture data. The heart rate signal may be deleted from the audio signal. The CPU 21 (a reproduction section) reproduces the heart sound based on the heart sound data 29 in accordance with the pace specified in the heart rate signal. The reproduced heart sound is inserted in the audio signal. The audio signal is transmitted to the headphone 31 through Bluetooth.

The motion picture is reproduced. The image signal serves to reproduce the image on the screen of the display panel 22. The audio signal serves to reproduce the sound from the headphone 31. The heartbeat of the character appearing in the image is reproduced in synchronization with the image reproduced based on the image signal. The human heart rate and the change of the heart rate reflect the human emotion. A person senses the emotion of the others by hearing or listening to the heart sound. The viewer of the motion picture is allowed to have identification with the character in the motion picture by hearing or listening to the heart sound. The viewer of the motion picture possibly enjoys more a realistic feeling through the motion picture than ever.

In the case where the motion picture data 46 is downloaded from the server 45, when the viewer selects a motion picture file based on the manipulation on the screen of the smartphone 15, the image signal and the audio signal are gained out of the motion picture data already stored in the memory 27. The CPU 21 separates the heart rate signal from the audio signal. The heart rate signal may be deleted from the audio signal. The CPU 21 (a reproduction section) reproduces the heart sound based on the heart sound data 29 in accordance with the pace specified in the heart rate signal. The reproduced heart sound is inserted in the audio signal. The audio signal is transmitted to the headphone 31 through Bluetooth.

The motion picture is reproduced. The image signal serves to reproduce the image on the screen of the display panel 22. The audio signal serves to reproduce the sound from the headphone 31. The heartbeat of the character appearing in the image is reproduced in synchronization with the image reproduced based on the image signal. The human heart rate and the change of the heart rate reflect the human emotion. A person senses the emotion of the others by hearing or listening to the heart sound. The viewer of the motion picture is allowed to have identification with the character in the motion picture by hearing or listening to the heart sound. The viewer of the motion picture possibly enjoys more a realistic feeling through the motion picture than ever.

Likewise, a user of the personal computer 17, the viewer, is allowed to start the software program 42. The personal computer 17 takes the heart sound data 47(43) in. The heart sound data 47 may be downloaded from the server 45 upon the installation of the software program 42 and stored in the large capacity storage device 41 of the personal computer 17, or may be downloaded from the server 45 upon reproduction of the heart sound and stored in the large capacity storage device 41 of the personal computer 17.

In the case of a so-called video streaming, where the motion picture data 46 is kept on the server, when the viewer selects a motion picture file based on the display on the screen of the display device 36, the server 45 transmits to the Internet 12 the motion picture data 46 containing an image data specifying an image, and a heart rate signal specifying the heart rate of a character appearing in the image. The motion picture data 46 is supplied to the CPU 33 of the personal computer 17 through the Internet 12 along the time axis. The CPU 33 separates the heart rate signal from the audio signal in the motion picture data 46. The heart rate signal may be deleted from the audio signal. The CPU 33 (a reproduction section) reproduces the heart sound based on the heart sound data 43 in accordance with the pace specified in the heart rate signal. The reproduced heart sound is inserted in the audio signal. The audio signal is transmitted to the headphone 44 through Bluetooth.

The motion picture is reproduced. The image signal serves to reproduce the image on the screen of the display device 36. The audio signal serves to reproduce the sound from the headphone 44. The heartbeat of the character appearing in the image is reproduced in synchronization with the image reproduced based on the image signal. The human heart rate and the change of the heart rate reflect the human emotion. A person senses the emotion of the others by hearing or listening to the heart sound. The viewer of the motion picture is allowed to have identification with the character in the motion picture by hearing or listening to the heart sound. The viewer of the motion picture possibly enjoys more a realistic feeling through the motion picture than ever.

In the case where the motion picture data 46 is downloaded from the server 45, when the viewer selects a motion picture file based on the display on the screen of the display device 36, the image signal and the audio signal are gained out of the motion picture data already stored in the large capacity storage device 41. The CPU 33 separates the heart rate signal from the audio signal. The heart rate signal may be deleted from the audio signal. The CPU 33 (a reproduction section) reproduces the heart sound based on the heart sound data 43 in accordance with the pace specified in the heart rate signal. The reproduced heart sound is inserted in the audio signal. The audio signal is transmitted to the headphone 44 through Bluetooth.

The motion picture is reproduced. The image signal serves to reproduce the image on the screen of the display device 36. The audio signal serves to reproduce the sound from the headphone 44. The heartbeat of the character appearing in the image is reproduced in synchronization with the image reproduced based on the image signal. The human heart rate and the change of the heart rate reflect the human emotion. A person senses the emotion of the others by hearing or listening to the heart sound. The viewer of the motion picture is allowed to have identification with the character in the motion picture by hearing or listening to the heart sound. The viewer of the motion picture possibly enjoys more a realistic feeling through the motion picture than ever.

Here, a vibration generating unit may be incorporated in the headphone 31, 44. The vibration generating unit may comprise a vibrating source configured to vibrate at a pace coinciding with the sound wave of the heartbeat. The vibrating source may use a vibrating motor, an electric actuator, an electromagnetic solenoid, a piezoelectric element or other microactuator, or the like. The magnitude of the vibration may be set enough to be sensed by the viewer when the vibration generating unit contacts the skin of the viewer. The vibrating source is configured to reproduce heartbeat by utilizing vibration. The vibration generating unit transmits the vibration to the human body based on the heart rate signal. In the case where the headphone 31, 44 is a bone conduction headphone, the bone transducer may be utilized to transmit the vibration. The bone transducer is configured to contact the skull of a viewer to vibrate like the diaphragm of a loudspeaker.

The CPU 21 of the smartphone 15 and the CPU 33 of the personal computer 17 receive the heart rate signal, gain the heart sound data 29, 43 respectively from the memory 27 and the large capacity storage device 41, and reproduce the heart sound based on the heart sound data 29, 43 in accordance with the pace specified in the heart rate signal. The heart rate signal is required to specify the pace of the heart rate. The heart rate signal is not required to specify the heart sound. It is not necessary to patch a microphone onto the chest to pick up the heart sound. An available common device such as a pulse sensor may be used to generate the heart rate signal. The heart sound can be reproduced in accordance with the heart rate in a simplified manner.

The imaging capturing apparatus 48 is used to generate the motion picture data 46. The motion picture is made based on the combination of the image specified in the image signal and the audio sound specified in the audio signal in the image capturing apparatus 48. The image capturing apparatus 48 comprises the mixing processing circuit 54 configured to output the audio signal containing the heart rate signal, generated at the pulse sensor apparatus 51, as sound mixed with the audio sound picked up from the microphone 53. Since the audio signal is mixed with the heart rate signal to create a motion picture, the heart rate signal is incorporated into a general format of a conventional motion picture. A peculiar format is not necessary to transmit the heart rate signal. The heart rate signal can be incorporated in a facilitated manner.

The heart sound may be embedded in the audio signal in advance to create a motion picture. The heart sound may be inserted into the audio signal in the motion picture data 46 at the server 45, or be inserted into the audio signal at the mixing processing circuit 54 of the image capturing apparatus 48. In either case, it is not necessary to hold the heart sound data 29, 43 at the reproduction terminals 13a, 13b. The reproduction terminals 13a, 13b are released from the mixing processing of the heart sound with the audio signal, so that the reproduction terminals 13a, 13b is allowed to enjoy a reduced burdensome.

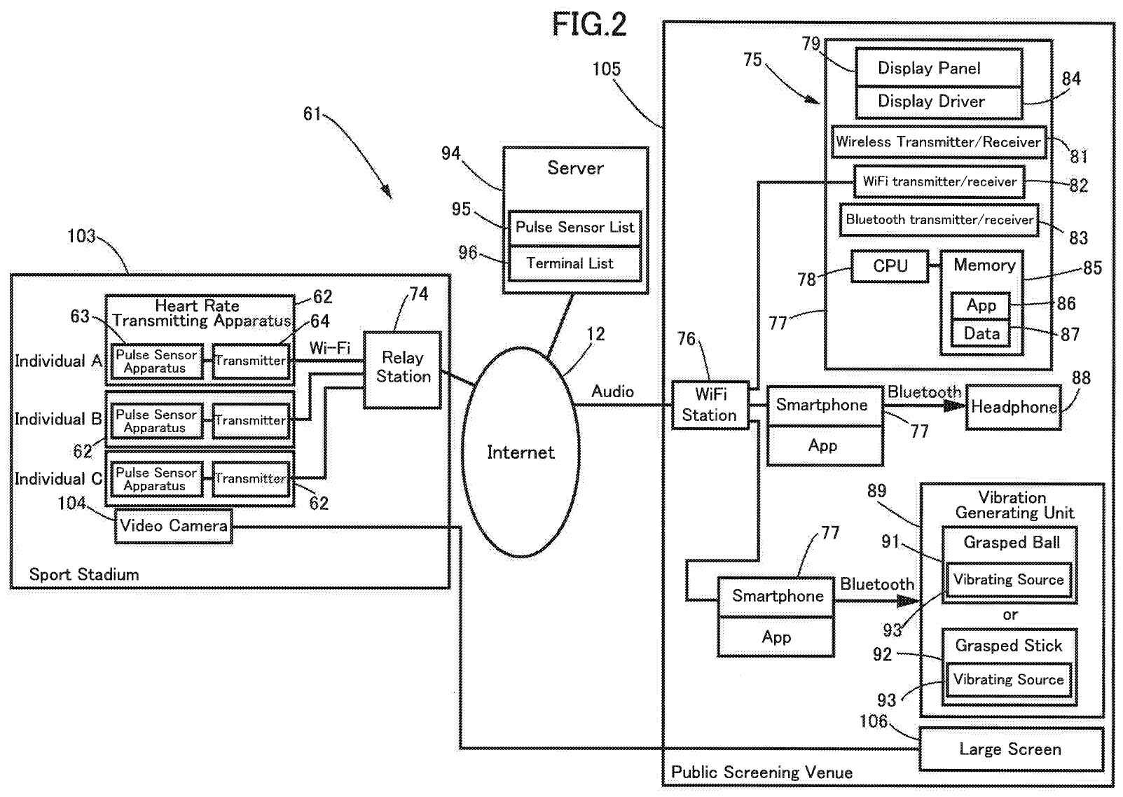

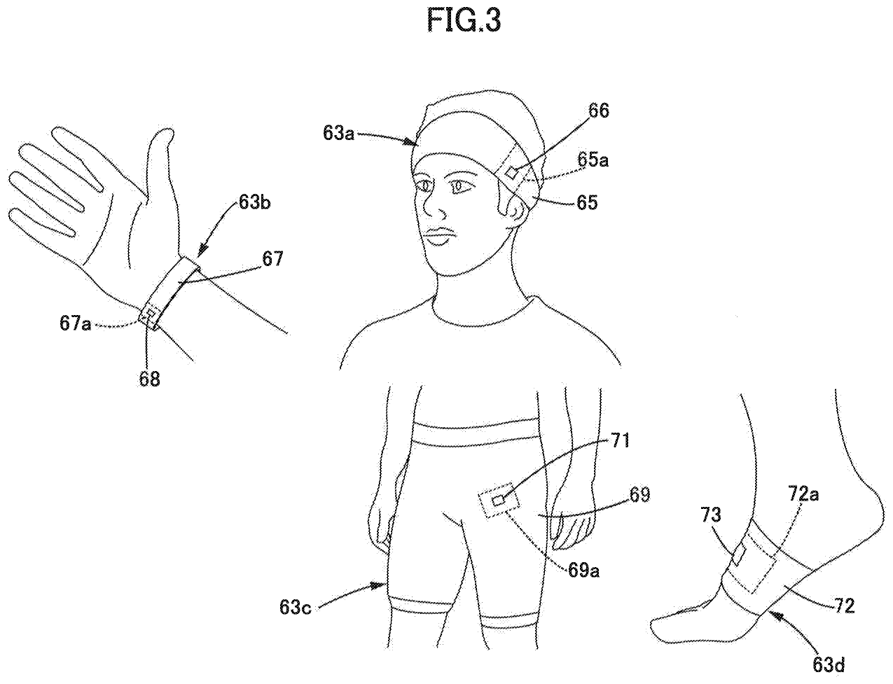

FIG. 2 schematically illustrates the structure of an emotion distributing system 61 according to a second embodiment. The emotion distributing system 61 includes heart rate transmitting apparatuses 62. The heart rate transmitting apparatus 62 comprises: a pulse sensor apparatus 63 configured to detect the pulse from an artery of an individual player of a team sport or an individual member of an idol group, hereinafter “an individual”, who wears the pulse sensor apparatus 63; and a transmitter 64 attached to the individual and configured to transmit the pulse via a radio wave. The pulse sensor apparatus 63 may take the form of a head band, a wrist band, an athletic supporter (briefs), an instep supporter, or the like. As depicted in FIG. 3, the pulse sensor apparatus 63a in the form of a head band may include, for example: a band body 65 worn around the head of the individual and having a contact area 65a contacting the temple of the individual; a pulse sensor 66 supported on the contact area 65a so as to detect the pulse from the superficial temporal artery of the individual. The pulse sensor apparatus 63b in the form of a wrist band may include, for example: a band body 67 worn around the wrist of the individual and having a contact area 67a contacting the inner surface of the wrist at a position near the little finger; and a pulse sensor 68 supported on the contact area 67a so as to detect the pulse from the radial artery. The pulse sensor apparatus 63c in the form of an athletic supporter may include, for example: a supporter body 69 as fitting briefs having a contact area 69a contacting the joint of a thigh; and a pulse sensor 71 supported on the contact area 69a so as to detect the pulse from the femoral artery of the individual. The pulse sensor apparatus 63d in the form of an instep supporter may include, for example: a supporter body 72 worn around the instep and the arch of a foot of the individual and having a contact area 72a contacting the instep of the individual; and a pulse sensor 73 supported on the contact area 72a so as to detect the pulse from the arteria dorsalis pedis of the individual.

The transmitter 64 is connected to the pulse sensor apparatus 63. The transmitter 64 is configured to process the output from the pulse sensor apparatus 63 and to transmit the heart rate signal toward a relay station 74. The relay station 74 is connected to the Internet 12. WiFi may be used to establish a communication between the transmitter 64 and the relay station 74. The heart rate signal is transmitted into the Internet 12 in this manner.

The emotion distributing system 61 includes a reproduction terminal (a heartbeat reproduction apparatus) 75 arbitrarily connectable to the Internet 12. Here, the reproduction terminal 75 comprises a smartphone 77 connected to the Internet 12 through a WiFi station 76, for example. The smartphone 77 may be connected to the Internet 12 through a mobile phone network.

The smartphone 77 includes: a CPU (central processing unit) 78 incorporated in a main enclosure; and a display panel 79 embedded in the front surface of the main enclosure. The display panel 79 defines a rectangular screen, for example, on the surface. The main enclosure encloses a wireless transmitter/receiver circuit 81 connected to a base station of a mobile phone network, a WiFi transmitter/receiver circuit 82 connected to the WiFi station 76, a Bluetooth transmitter/receiver circuit 83, a display driver 84 configured to control the display on the screen, and other electronic circuit, all connected to the CPU 78.

The CPU 78 is configured to receive a heart rate data from the Internet through the WiFi transmitter/receiver circuit 82 (or the wireless transmitter/receiver circuit 81). The heart rate signal may comprise an audio signal specifying the audio sound. Here, the heart rate signal is designed to specify the pace of the heart rate. The pace of the heartbeat may be expressed based on the intervals of the pulse signals, other rhythmic signal, or the like, for example. The pace of the heartbeat may be defined by the intervals between the first sounds or the intervals between the second sounds, for example.

A memory (storage section) 85 is connected to the CPU 78. The memory 85 is configured to keep a mobile application program (smartphone application) 86 and a heart sound data 87. The heart sound data 87 is designed to specify the sound waves of heart sounds. The heart sound data 87 may also function as a heartbeat data specifying heartbeat. The CPU 78 is configured to execute the mobile application program 86. The execution of the mobile application program 86 enables the CPU 78 (a receiving section) to receive the heart rate signal, to gain the heart sound data 87 from the memory 85, and to reproduce the heart sound based on the heart sound data 87 in accordance with the pace specified in the heart rate signal.

A headphone 88 is connected to the smartphone 77. A cable may be used to connect the headphone 88 to the smartphone 77, or any wireless means is used to connect the headphone 88 to the smartphone 77. Here, Bluetooth may be used to connect the headphone 88 to the smartphone 77. The heart rate signal is supplied to the headphone 88 from the CPU 78 through the Bluetooth transmitter/receiver circuit 83. The loudspeaker of the headphone 88 emits the sound waves in the air in response to the action of the diaphragm, for example. The headphone 88 is worn on the head of a person. The headphone 88 may preferably be a bone conduction headphone.

The CPU 78 is configured to decode the heart rate signal from the audio signal. The CPU 78 (a reproduction section) is configured to reproduce the heart sound based on the heart rata signal. The ears of a person receive the heart sound of a person from the headphone 88.

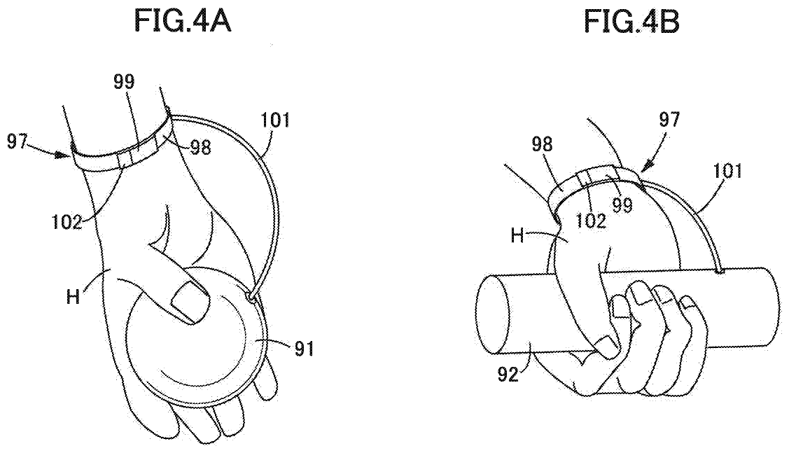

A vibration generating unit 89 may be connected to the smartphone 77. The vibration generating unit 89 comprises a grasped ball 91 (FIG. 4A) or a grasped stick 92 (FIG. 4B) both connected to the smartphone 77 through a cable or wireless means. As depicted in FIG. 4A, the grasped ball 91 is formed in a spherical shape having a size suitable to be grasped. As depicted in FIG. 4B, the grasped stick 92 is formed in a bar-shape having a thickness suitable to be grasped. A vibrating source 93 is embedded in the grasped ball 91 and the grasped stick 92. The vibrating source 93 is configured to vibrate at a pace coinciding with the sound wave of the heartbeat. The vibrating source may use a vibrating motor, an electric actuator, an electromagnetic solenoid, a piezoelectric element or other microactuator, or the like. The magnitude of the vibration may be set enough to be sensed by the hand H grasping the grasped ball 91 or the grasped stick 92. The vibrating source 93 is configured to reproduce heartbeat by utilizing vibration. The vibration generating unit 89 transmits the vibration to the hand H based on the heart rate signal.

The emotion distributing system 61 includes a server 94 connected to the Internet 12. The server 94 is configured to hold: a pulse sensor list 95 designed to relate the heart rate signals to the corresponding individuals; and a terminal list 96 designed to relate the reproduction terminals 75 to the corresponding individuals. The pulse sensor list 95 and the terminal list 96 in combination serve to relate the heart rate signal to the individual reproduction terminals 75. The individual reproduction terminal 75 is in this manner designed to receive a corresponding heart rate signal. The heart sound is reproduced based on the heart rate signal.

A rental system may be combined with the vibration generating unit 89. The rental system comprises a wristband connected to the grasped ball 91 or the grasped stick 92. The wristband is worn around the wrist of a person, for example. The wristband 97 includes: a strip body 98 worn around the wrist; a locking mechanism 99 fixed to the opposite ends of the strip body 98 in the longitudinal direction and switched over between a coupled status and a decoupled status by an electric power; and a wire 101 designed to couple the grasped ball 91 or the grasped stick 92 with the corresponding wristband 97. When the locking mechanism 99 couples the opposite ends of the strip body 98, the wristband 97 encircles the wrist of a user with a size in circumference enough to prevent the hand H from passing through. A so-called locked state is established. The establishment of the locked state enables an unremovable attachment of the wristband 97 to the wrist of the person. When the locking mechanism 99 decouples the opposite ends of the strip body 98, the wristband 97 transitions to the opened state from the encircled state so as to separate from the wrist of the person. A so-called unlocked state is established.

A transmitter/receiver circuit 102 is connected to the locking mechanism 99. The transmitter/receiver circuit 102 is configured to supply electric power to the locking mechanism 99 in response to the reception of a predetermined radio wave so as to establish the unlocked state of the locking mechanism 99. The locking mechanism 99 can be automatically unlocked in response to the reception of the predetermined radio wave. The transmitter/receiver 102 may exchange signals with an exterior entry/exit management system. For example, the transmitter/receiver circuit 102 may be allowed to transmit a signal to the entry/exit management system upon the exit of a person from a venue. The entry/exit management system is allowed to prevent the exit of the person out of the venue in response to the reception of the signal. The rented grasped ball 91 or stick 92 may be prevented from being taken away out of the venue.

Next, explanation will be made on the action of the emotion distributing system 61. The emotion distributing system 61 may be utilized in a public screening of a team sport, for example. The public screening employs a video camera 104 capable of capturing a real-time motion images of a match or a game of the team sport held at a sport stadium or arena 103. The video camera 104 generates an image signal. The image signal is supplied to a large screen 106 at a public screening venue 105. The corresponding audio sound may be reproduced at the public screening venue 105 based on the audio signal in synchronization with the image.

The audience at the public screening venue 105 taps the screen of the smartphone 77 to start the mobile application program 86, for example. When the audience selects an athlete based on the manipulation on the screen of the smartphone 77, the server 94 registers the identifier of the smartphones 77 in the terminal list 96 for the individual athletes. The server 94 holds the identifier of the pulse sensor apparatus 63 assigned to the individual athlete in the pulse sensor list 95. The athlete wears the pulse sensor apparatus 63 corresponding to the designated identifier. The individual smartphone 77 is in this manner related to the designated pulse sensor apparatus 63. The audience may be charged through the smartphone 77 for a price for the relating processes. The charge may be applied when the mobile application program 86 has been downloaded, or when the athlete has been selected.

The mobile application program 86 takes the heart sound data 87 in. The heart sound data 87 may be downloaded from the server 94 upon the installation of the mobile application program 86 and stored in the memory 85 of the smartphone 77, or may be downloaded from the server 94 upon the reproduction of the heart sound and stored in the memory 85 of the smartphone 77. Here, the heart sound data 87 is registered in the server 94 for the individual athletes. The smartphone 77 receives the download of the heart sound data 87 assigned to the athlete selected at the smartphone 77. The heart sound data 87 may be established based on a beforehand actual measurement for the individual athletes.

The heart sound data 87 may be a single heart sound data common to all the athletes, or may be selected from a group of a common heart sound data and a unique heart sound data set including heart sound data unique to the individual athletes. In the latter case, the common heart sound data may beforehand be incorporated in the mobile application program 86 as the standard data while the heart sound data unique to the selected athlete may be downloaded to the memory 85 upon the payment of the charge.

The headphone 88 is worn on the head of a person. A communication path is established between the headphone 88 and the smartphone 77 through Bluetooth, for example. The audience may grasp the grasped ball 91 or the grasped stick 92 in addition to (or in place of) the headphone 88. A communication path is established between the grasped ball 91 or stick 92 and the smartphone 77 through Bluetooth.

The pulse of the individual athlete is measured straightaway. The relay station 74 instantly receives the heart rate signal from the transmitter 64. The server 94 instantly gains the heart rate signal from the individual pulse sensor apparatus 63 through the Internet 12. The server 94 instantly transmits the heart rate signal, related to the smartphone 77 based on the terminal list 96, to the designated smartphone 77. The CPU 78 of the individual smartphone 77 receives the heart rate signal. The CPU 78 (a reproduction section) straightaway reproduces the heart sound based on the heart sound data 87 in accordance with the pace specified in the heart rate signal. The reproduced heart sound is transmitted as an audio signal to the headphone 88 through Bluetooth. The CPU 78 instantly reproduces the heart sound based on the heart rate signal.

The pulse sensor apparatus 63 is individually attached to the athletes. The smartphone 77 is allowed to receive the heart rate signal of the athlete previously related to the smartphone 77. The individual smartphone 77 reproduces the heart sound based on the heart rate signal related to the smartphone 77. A particular athlete among the athletes is assigned to the individual smartphone 77. The heart sound of the particular athlete is reproduced at the corresponding smartphone 77. The audience is allowed to enjoy identification with the favorite athlete by hearing or listening to the heart sound of the favorite athlete.

For example, the pulse sensor apparatus 63d in the form of an instep supporter comprises: a supporter body 72 worn around the instep and the arch of a foot of the individual and having a contact area 72a contacting the instep of the individual; and a pulse sensor 73 supported on the contact area 72a so as to detect the pulse from the arteria dorsalis pedis of the individual. The pulse sensor apparatus 63d is attached to the foot in front of the ankle for detecting pulse. The pulse sensor apparatus 63d does not hinder the movement of the foot around the ankle. Moreover, the human body is relatively rigid at the arch of the foot, so that the pulse sensor apparatus 63d is sufficiently prevented from displacement. A wearer may feel a comfortable fitness. The pulse sensor 73 may operate to detect the pulse from the arteria dorsalis pedis with an unconscious wearing of the pulse sensor apparatus 63d.

The distribution of the heart rate signal may be limited inside the public screening venue 105, or any other area or geographic region. Any of the WiFi stations 76 may be designated to transmit the heart rate signal for limiting the geographic region. The emotion distributing system 61 may be utilized to live stream the image of an individual sport, a single idol, or any other individual activity, in addition to the team sport or the idol group.

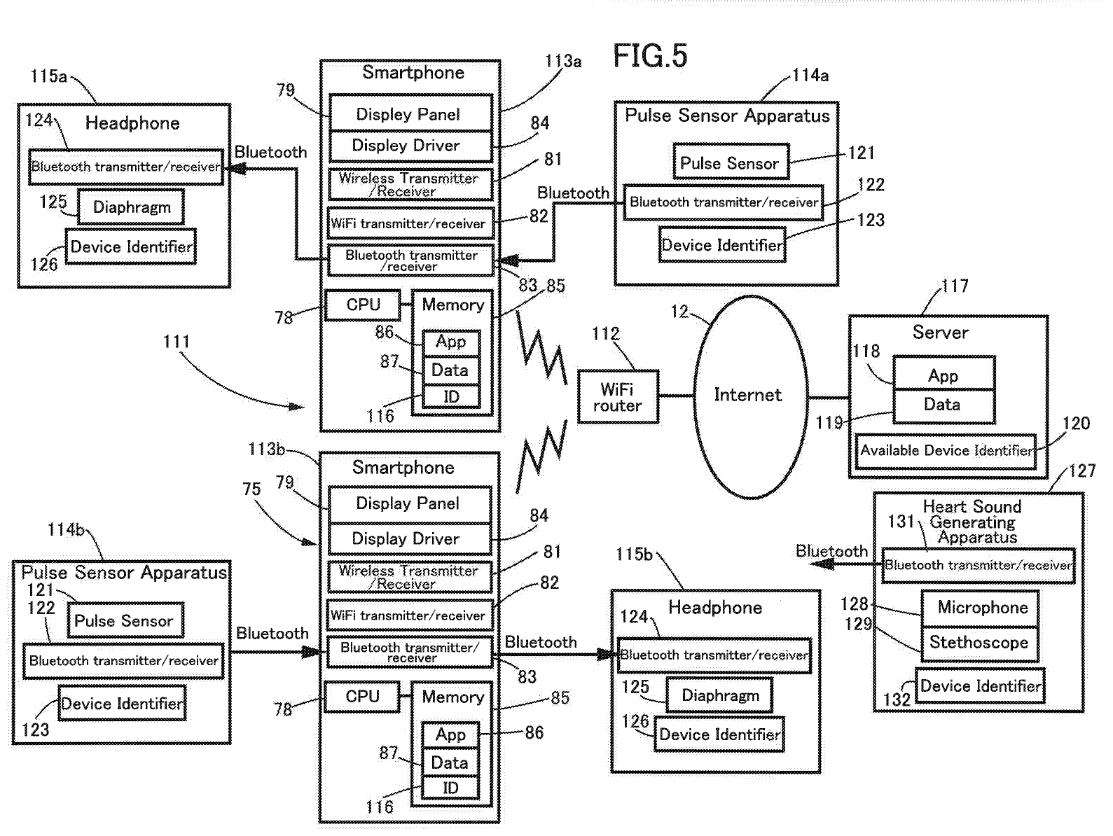

FIG. 5 schematically illustrates the structure of a heartbeat reproduction apparatus 111 according to a third embodiment. The heartbeat reproduction apparatus 111 comprises: smartphones (the body of a reproduction terminal) 113a, 113b connected to the Internet 12 through a WiFi router 112 or a mobile phone network, for example; pulse sensor apparatuses 114a, 114b connected respectively to the smartphones 113a, 113b and configured to detect the pulse from the artery of an individual; and headphones 115a, 115b connected respectively to the smartphones 113a, 113b and configured to emit the sound waves in the air in response to the action of the diaphragm based on an audio signal. A cable may be used to connect the pulse sensor apparatuses 114a, 114b and the headphones 115a, 115b, respectively, to the smartphones 113a, 113b, or any wireless means may be used to connect the pulse sensor apparatuses 114a, 114b and the headphones 115a, 115b, respectively, to the smartphones 113a, 113b. Here, Bluetooth is used to establish the connection.

The smartphones 113a, 113b have the structure identical to that of the aforementioned smartphone 77. Specifically, the individual smartphone 113a, 113b includes: a CPU (central processing unit) 78 incorporated in a main enclosure; and a display panel 79 embedded in the front surface of the main enclosure. The display panel 79 defines a rectangular screen, for example, on the surface. The main enclosure encloses a wireless transmitter/receiver circuit 81 connected to a base station of a mobile phone network, a WiFi transmitter/receiver circuit 82 connected to the WiFi router 112, a Bluetooth transmitter/receiver circuit 83, a display driver 84 configured to control the display on the screen, and other electronic circuit, all connected to the CPU 78.

A memory (storage section) 85 is connected to the CPU 78. The memory 85 is configured to keep a mobile application program (smartphone app) 86, a heart sound data 87 and an ID data 116. The mobile application program 86, the heart sound data 87 and the ID data 116 are downloaded from the Internet 12 through the WiFi transmitter/receiver circuit 82 (or the wireless transmitter/receiver circuit 81), for example. The heart sound data 87 is designed to specify the sound waves of heart sounds. The ID data 116 is designed to specify an available device identifier list peculiar to the mobile application program 86. The available device identifier list includes identifiers assigned respectively to individual models or individual product makers of the pulse sensor apparatus 114a, 114b and the headphone 115a, 115b, for example. The CPU 78 is configured to execute the mobile application program 86. The execution of the mobile application program 86 enables the CPU 78 (a receiving section) to receive the heart rate signal from the pulse sensor apparatus 114a, 114b, to gain the heart sound data 87 from the memory 85, and to reproduce the heart sound based on the heart sound data 87 in accordance with the pace specified in the heart rate signal.

A server 117 is connected to the Internet 12. A mobile application program 118, a heart sound data 119 and an available device identifier list 120 are registered in the server 117. The mobile application program 118, the heart sound data 119 and the available device identifier list 120 are downloaded from the server 117 to the individual smartphone 113a, 113b through the Internet 12 in response to the operation of the smartphone 113a, 113b. The available device identifier list 120 is stored in the memory 85 as the ID data 116. A user of the smartphone 113a, 113b may be charged through the smartphone 113a, 113b for a price of the download of the mobile application program 118, the heart sound data 119 and the available device identifier list 120.

The pulse sensor apparatus 114a, 114b comprises: a pulse sensor 121 configured to detect the pulse from the artery so as to output a heart rate signal specifying the heart rate of an individual; a Bluetooth transmitter/receiver circuit 122 connected to the pulse sensor 121 and configured to transmit the heart rate signal through Bluetooth; and a memory 123 connected to the Bluetooth transmitter/receiver circuit 122 and configured to keep a device identifier unique to the individual pulse sensor apparatus 114a, 114b supplied to the smartphone 113a, 113b. The pulse sensor apparatus 114a, 114b may take the form of a head band, a wrist band, an athletic supporter (briefs), an instep supporter, or the like. The Bluetooth transmitter/receiver circuit 122 and the memory 123 may be supported on the band body 65, 67 or the supporter body 69, 72 along with the pulse sensor 121. The pulse sensor 121, the Bluetooth transmitter/receiver circuit 122 and the memory 123 may be assembled on a single card or in a single unit. The device identifier may be registered in the memory 123 at a factory of the pulse sensor apparatus 114a, 114b. The memory 123 may be a flash memory, a read only memory (ROM), or the like, for example.

The heart rate signal may comprise an audio signal specifying the sound. Here, the heart rate signal is designed to specify the pace of the heartbeat. The pace of the heartbeat may be expressed based on the intervals of the pulse signals, other rhythmic signal, or the like, for example. The pace of the heartbeat may be defined by the intervals between the first heart sounds or the intervals between the second heart sounds, for example.

The CPU 78 of the smartphone 113a, 113b is configured to compare the device identifier gained from the pulse sensor apparatus 114a, 114b with a connectable device identifier in the ID data 116 upon the establishment of a communication path via Bluetooth (upon the registration of the device identifier). If the device identifier of the pulse sensor apparatus 114a, 114b is confirmed upon the comparison, the heart rate signal is transmitted to the smartphone 113a, 113b from the pulse sensor apparatus 114a, 114b. Unless the device identifier of the pulse sensor apparatus 114a, 114b is confirmed, the heart rate signal is not supplied to the smartphone 113a, 113b.

The headphone 115a, 115b comprises: a Bluetooth transmitter/receiver circuit 124 configured to establish a communication path with the smartphone 113a, 113b and to receive the audio signal from the smartphone 113a, 113b; diaphragm 125 connected to the Bluetooth transmitter/receiver circuit 124 and configured to vibrate in response to the audio signal supplied; and a memory 126 connected to the Bluetooth transmitter/receiver circuit 124 and configured to keep the device identifier unique to the headphone 115a, 115b supplied to the smartphone 113a, 113b. The device identifier may be registered in the memory 126 at a factory of the headphone 115a, 115b. The memory 126 may be a flash memory, a read only memory (ROM), or the like, for example. The sound waves of the heart sound is emitted in the air in response to the action of the diaphragm 125. The headphone 115a, 115b is worn on the head of a person. The headphone 115a, 115b may preferably be a bone conduction headphone.

The CPU 78 of the smartphone 113a, 113b is configured to compare the device identifier gained from the headphone 115a, 115b with a connectable device identifier in the ID data 116 upon the establishment of a communication path via Bluetooth (upon the registration of the device identifier). If the device identifier of the headphone 115a, 115b is confirmed upon the comparison, the audio signal is transmitted to the headphone 115a, 115b from the smartphone 113a, 113b. Unless the device identifier of the headphone 115a, 115b is confirmed, the audio signal is not supplied to the headphone 115a, 115b.

The heartbeat reproduction apparatus 111 may include a heart sound generating apparatus 127 connected to the smartphone 113a, 113b and configured to detect the heart sound from an individual so as to output an audio signal specifying the heart sound. The heart sound generating apparatus 127 includes: a microphone 128 configured to gain the sound waves of the heartbeat from the human body; a stethoscope 129 connected to the microphone 128 and configured to detect the heart sound from any of the aortic valve area, the pulmonary valve area, the tricuspid valve area, the mitral valve area and the Erb's point and to transmit the amplified heart sound to the microphone 128; a Bluetooth transmitter/receiver circuit 131 connected to the microphone 128 and configured to transmit the audio signal of the heartbeat through Bluetooth; and a memory 132 connected to the Bluetooth transmitter/receiver circuit 131 and configured to keep a device identifier unique to the heart sound generating apparatus 127 supplied to the smartphone 113a, 113b. The device identifier may be registered in the memory 132 at a factory of the heart sound generating apparatus 127. The memory 132 may be a flash memory, a read only memory (ROM), or the like, for example.

The microphone 128 is configured to output an electric signal specifying the sound waves of the heartbeat, namely a heart sound data. The pace of the heartbeat may arbitrarily be set. The heart sound data may be prepared for various paces. The heart sound data may be stored in the memory 85 of the smartphone 113a, 113b as the heart sound data 87. The heart sound data 87 may be grouped for individual persons.

The CPU 78 of the smartphone 113a, 113b is configured to compare the device identifier gained from the heart sound generating apparatus 127 with a connectable device identifier in the ID data 116 upon the establishment of a communication path via Bluetooth (upon the registration of the device identifier). If the device identifier of the heart sound generating apparatus 127 is confirmed upon the comparison, the audio signal of the heartbeat is transmitted to the smartphone 113a, 113b from the heart sound generating apparatus 127. Unless the device identifier of the heart sound generating apparatus 127 is confirmed, the audio signal of the heart sound generating apparatus 127 is not supplied to the smartphone 113a, 113b.

Next, explanation will be made on the action of the heartbeat reproduction apparatus 111. A user of the smartphone 113a, 113b downloads the mobile application program 118 and the heart sound data 119 into the smartphone 113a, 113b, for example. The available device identifier list 120 is also downloaded along with the mobile application program 118. An instruction image for download manipulation may be displayed on the screen of the smartphone 113a, 113b. The user then installs the mobile application program 118 in the smartphone 113a, 113b. The heart sound data 119 and the available device identifier list 120 are registered into the mobile application program 118 upon the installation of the mobile application program 118. The mobile application program 86, the heart sound data 87 and the ID data 116 are stored in the memory 85.

A first user (a first person) connects the pulse sensor apparatus 114a and the headphone 115a to the smartphone 113a. The first user taps the screen of the smartphone 113a to start the mobile application program 86, for example, for the connection of the pulse sensor apparatus 114a and the headphone 115a. When the pulse sensor apparatus 114a is connected to the smartphone 113a (the body of a reproduction terminal), the CPU 78 of the smartphone 113a is configured to gain the device identifier of the pulse sensor apparatus 114a through Bluetooth. The CPU 78 is configured to then compare the device identifier of the pulse sensor apparatus 114a with the available device identifier list registered in the memory 85 as the ID data so as to confirm whether or not the pulse sensor apparatus 114a is registered or not. If the registration of the pulse sensor apparatus 114a has been confirmed, a communication path of Bluetooth is established between the smartphone 113a and the pulse sensor apparatus 114a.

Likewise, when the headphone 115a is connected to the smartphone 113a, the CPU 78 of the smartphone 113a is configured to gain the device identifier of the headphone 115a through Bluetooth. The CPU 78 is configured to then compare the device identifier of the headphone 115a with the available device identifier list registered in the memory 85 as the ID data 116 so as to check whether or not the headphone 115a is registered or not. If the registration of the headphone 115a has been confirmed, a communication path of Bluetooth is established between the smartphone 113a and the headphone 115a. A second user (a second person) of the smartphone 13b likewise connects the other pulse sensor apparatus 114b and the other headphone 115b to the smartphone 113b.

The first user wears the (first) headphone 115a connected to the smartphone 113a. The second user wears the (first) pulse sensor apparatus 114a connected to the smartphone 113a. When the reproduction of the heart sound is started, the heart rate signal is transmitted to the smartphone 113a from the pulse sensor apparatus 114a through Bluetooth. The CPU 78 of the smartphone 113a is allowed to straightaway receive the heart rate signal from the pulse sensor apparatus 114a. The CPU 78 (a reproduction section) is configured to instantly reproduce the heart sound based on the heart sound data 87 in accordance with the pace specified in the heart rate signal. The heart sound is transmitted to the headphone 115a as the audio signal through Bluetooth. The CPU 78 is configured to instantly reproduce the heart sound based on the heart rate signal. The second user is allowed to enjoy identification with the first user by hearing or listening to the heart sound of the first user.

In this case, the second user may wear the (second) headphone 115b connected to the smartphone 113b, while the first user may wear the (second) pulse sensor apparatus 114b connected to the smartphone 113b. When the reproduction of the heart sound is started, the heart rate signal is transmitted to the smartphone 113b from the pulse sensor apparatus 114b through Bluetooth. The CPU 78 of the smartphone 113b is allowed to straightaway receive the heart rate signal from the pulse sensor apparatus 114b. The CPU 78 (a reproduction section) is configured to instantly reproduce the heart sound based on the heart sound data 87 in accordance with the pace specified in the heart rate signal. The heart sound is transmitted to the headphone 115b as the audio sound through Bluetooth. The CPU 78 is configured to instantly reproduce the heart sound based on the heart rate signal. The first and the second users are allowed to enjoy identification with each other by hearing or listening to the heart sound of the other.

A method of reproducing the heart sound according to the embodiment includes causing the CPU 78 to gain the device identifier of the pulse sensor apparatus 114a, 114b when the pulse sensor apparatus 114a, 114b is connected to the smartphone 113a, 113b as the body of the reproduction terminal. The CPU 78 is caused to compare the device identifier gained with the available device identifier list so as to check whether or not the pulse sensor apparatus 114a, 114b is registered. If the registration of the pulse sensor apparatus 114a, 114b has been confirmed, the CPU 78 is allowed to gain the heart rate signal from the pulse sensor apparatus 114a, 114b. Unless the device identifier of the pulse sensor apparatus 114a, 114b is confirmed, the CPU 78 does not gain the heart rate signal from the pulse sensor apparatus 114a, 114b. The heart rate signal can be gained solely from the designated pulse sensor apparatus 114a, 114b. The pulse sensor apparatus 114a, 114b connectable is in this manner distinguished from the other pulse sensor apparatuses.

A method of reproducing the heart sound according to the embodiment includes causing the CPU 78 to gain the device identifier of the headphone 115a, 115b when the headphone 115a, 115b is connected to the smartphone 113a, 113b as the body of the reproduction terminal. The CPU 78 is caused to compare the device identifier of the headphone 115a, 115b with the available device identifier list so as to check whether or not the headphone 115a, 115b is registered. If the registration of the headphone 115a, 115b has been confirmed, the CPU 78 is allowed to output the audio signal of the heart sound to the headphone 115a, 115b. Unless the device identifier of the headphone 115a, 115b is conformed, the CPU 78 denies the output of the audio signal of the heart sound to the headphone 115a, 115b. The audio signal can be output solely to the designated headphone 115a, 115b. The headphone 115a, 115b connectable is in this manner distinguished from the other headphones.

The user of the smartphone 113a may wear the pulse sensor apparatus 114a and the headphone 115a both connected to the smartphone 113a. The user of the smartphone 113a can hear or listen to his/her own heartbeat through the headphone 115a. The user of the smartphone 113a become conscious about his/her own heartbeat. The user may train the control of emotion, for example, by hearing or listening to his/her own heart sound.

The wearer of the pulse sensor apparatus 114a, 114b may register his/her own heart sound into the mobile application program 86 by utilizing the heart sound generating apparatus 127. The microphone 128 is configured to output an electric signal specifying the sound waves of the heartbeat, namely a heart sound data. The pace of the heartbeat may arbitrarily be set. The heart sound data may be prepared for various paces. The heart sound data may be stored in the memory 85 of the smartphone 113a, 113b as the heart sound data 87.

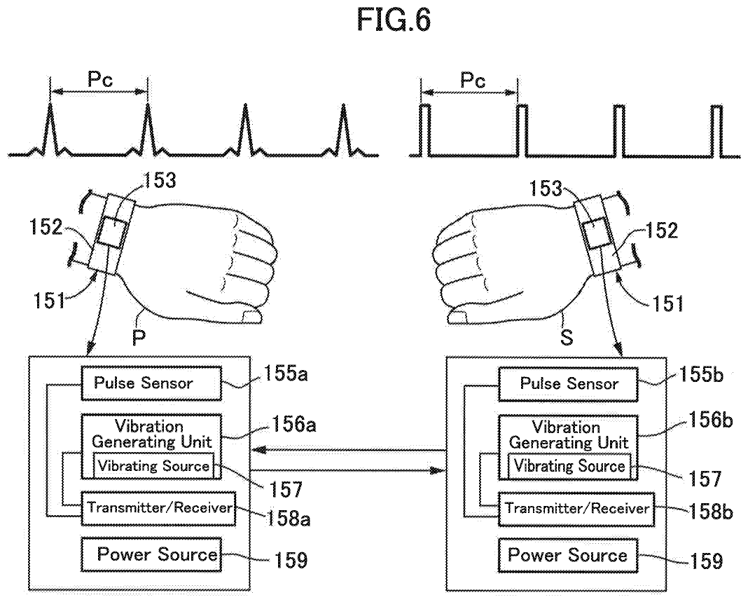

FIG. 6 schematically illustrates the structure of a heartbeat reproduction apparatus 151 according to a fourth embodiment. The heartbeat reproduction apparatus 151 takes the form of a wrist band. Specifically, the heartbeat reproduction apparatus 151 includes: a band body 152; and a heartbeat reproduction unit 153 incorporated in the band body 152. The band body 152 may be made of an elastic rubber, or a leather strip, a cloth strip, a plastic strip or a metal strip, adjustable in length with a buckle, or the like. The band body 152 is worn around the wrist of a user P, S. The heartbeat reproduction unit 153 is preferably brought in a close contact with the skin of the user P, S.

The individual heartbeat reproduction unit 153 includes a pulse sensor 155a, 155b. The pulse sensor 155a, 155b is configured to detect the pulse from the wrist. Every pulse is identified. The pulse sensor 155a, 155b is configured to output a heart rate signal specifying the heart rate based on the detected pulse. The heart rate signal is designed to specify the pace of the heart rate. The pace of the heart rate may be expressed based on the intervals of the pulse signals, other rhythmic signal, or the like, for example. The pace of the heartbeat may be defined by the intervals between the first heart sounds or the intervals between the second heart sounds, for example.

The heartbeat reproduction unit 153 comprises a vibration generating unit 156a, 156b. The vibration generating unit 156a, 156b includes a vibrating source 157 configured to vibrate in accordance with the heart rate specified in the heart rate signal. The vibrating source 157 may employ a vibrating motor, an electric actuator, an electromagnetic solenoid, a piezoelectric element or other microactuator, or the like. The magnitude of the vibration may be set enough to be sensed by the user P, S when the vibration generating unit 156a, 156b contacts the skin of the user P, S. The vibrating source 157 is configured to reproduce heartbeat by utilizing vibration.

The heartbeat reproduction unit 153 comprises a transmitter/receiver circuit 158a, 158b. The transmitter/receiver circuit 158a, 158b is connected to the pulse sensor 155a, 155b and the vibration generating unit 156a, 156b. The transmitter/receiver circuit 158a(158b) is configured to receive the heart rate signal from the transmitter/receiver circuit 158b(158a) incorporated in another heart reproduction apparatus 151, hereinafter the “paired apparatus 151”. The heart rate signal is supplied from the transmitter/receiver circuit 158a(158b) to the vibration generating unit 156a(156b) incorporated in the identical heart reproduction apparatus 151, hereinafter “identical apparatus 151”. In this case, the transmitter/receiver circuit 158a(158b) functions as a receiver circuit. A first channel of a communication path is established between the transmitter/receiver circuit 158a(158b) and the transmitter/receiver circuit 158b(158a) in the paired apparatus 151 for receiving the heart rate signal. Bluetooth, IrDA, WiFi, or other radio communication standard, for example, may be utilized to establish the communication path.

The transmitter/receiver circuit 158a(158b) is configured to receive the pulse sensor 155a(155b) incorporated in the identical apparatus 151. The heart rate signal is then transmitted from the transmitter/receiver circuit 158a(158b) to the transmitter/receiver circuit 158b(158a) of the paired apparatus 151. In this case, the transmitter/receiver circuit 158a(158b) functions as a transmitter circuit. A second channel, distinguished from the first channel, of the communication path may be established between the transmitter/receiver circuit 158a(158b) and the transmitter/receiver circuit 158b(158a) of the paired apparatus 151 for transmitting the heart rate signal.

The heartbeat reproduction unit 153 comprises a power source 159. The power source 159 is connected to the pulse sensor 155a(155b), the vibration generating unit 156a(156b) and the transmitter/receiver circuit 158a(158b). The power source 159 is configured to supply electricity to the pulse sensor 155a(155b), the vibration generating unit 156a(156b) and the transmitter/receiver circuit 158a(158b). The power source 159 may employ a primary battery, a secondary battery, or the like. Alternatively, the electricity may be supplied to the power source 159 through a radio communication.

The heartbeat reproduction apparatus 151 is used in a pair. The heartbeat reproduction apparatus 151 is worn around the wrist of the individual user P, S. The pulse sensor 155a detects the pulse of the user P. The pulse of the user P reflects the heart rate. The transmitter/receiver circuit 158a serves to transmit a heart rate signal specifying the heart rate to the transmitter/receiver circuit 158b. The transmitter/receiver circuit 158b supplies the heart rate signal to the vibration generating unit 156b. The vibration generating unit 156b is configured to generate vibration (or impact) at a predetermined interval in accordance with the heart rate signal. The user S is thus allowed to feel the heart rate (heartbeat) of the user P. Likewise, the user P is allowed to feel the heart rate (heartbeat) of the user S. The users P, S become conscious about the heartbeat of the other.

The transmitter/receiver circuit 158a, 158b may have a limited range of radio communication. If one of the transmitter/receiver circuits 158a(158b) is located off the limited range of the radio communication of the other transmitter/receiver circuit 158b(158a), exchange of the heart rate signal is terminated. If the user P(S) gets remoter from the user S(P), the reproduction of the heartbeat terminates. The limitation of the special range of the radio communication enables a reduced consumption of the electricity. When a communication channel is commonly assigned to the heartbeat reproduction apparatuses 151, the limitation of the range of the radio communication enables exchange of the heart rate signal solely between the designated pairs of users P, S among many users.

The heart rate signal may be transmitted from the pulse sensor 155a(155b) directly to the vibration generating unit 156a(156b) of the identical apparatus 151. The user P, S is allowed to become conscious about his/her own heart rate. The user P, S may recognize his/her own heart rate during the observation inside himself/herself. In this case, the heartbeat reproduction apparatus 151 may be dedicated to reproduction of own heart rate (heartbeat) by omitting the transmitter/receiver circuit 158a(158b).

It should be noted that the heartbeat reproduction apparatus 151 may take the form of an arm band, a ring, a sticker adhered to the skin of a user, or the like. The heartbeat reproduction unit 153 may be incorporated in a watch, an earphone, glasses, a belt, or other accessory, and the like.

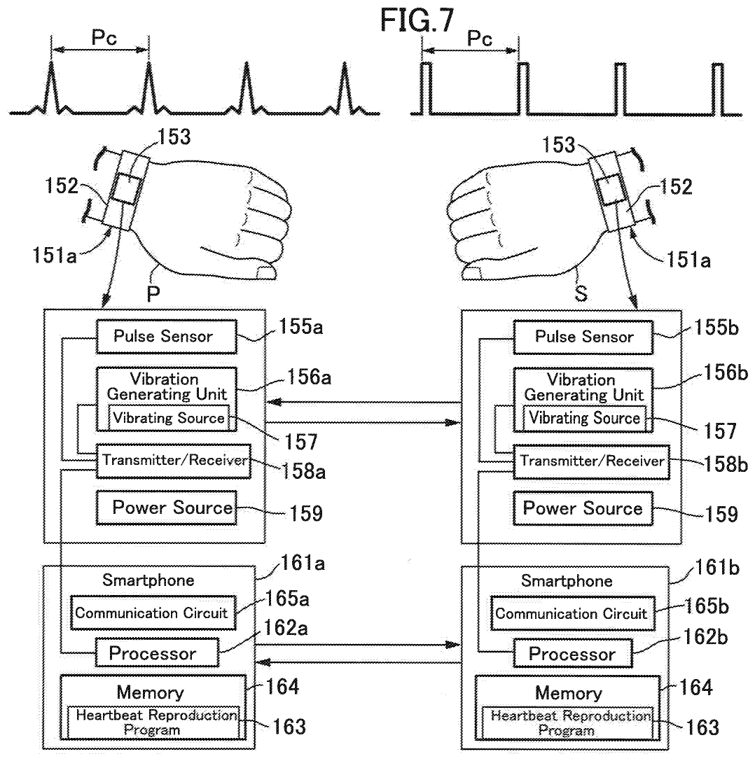

FIG. 7 schematically illustrates the structure of a heartbeat reproduction apparatus 151a according to a fifth embodiment. The heartbeat reproduction apparatus 151a is connected to smartphone 161a, 161b. The transmitter/receiver circuit 158a, 158b of the heartbeat reproduction apparatus 151a is connected to the processor 162a, 162b of the individual smartphone 161a, 161b. A connector cable may be utilized to connect the transmitter/receiver circuit 158a, 158b to the processor 162a, 162b, or a radio communication such as Bluetooth may be utilized to connect the transmitter/receiver circuit 158a, 158b to the processor 162a, 162b. The processor 162a, 162b is configured to execute a software program 163 to run the application (heartbeat reproducing software). The software program 163 is stored in a memory 164, for example. The software program 163 may be downloaded into the memory 164 from a server via the Internet 12, for example.

When the software program 163 is executed to establish a heartbeat reproduction software, the processor 162a, 162b is configured to gain the heart rate signal from the pulse sensor 155a, 155b. The heart rate signal is then transmitted from a communication circuit 165a(165b) of the smartphone 161a(161b) to the paired smartphone 161b(161a). The processor 162b(162a) of the paired smartphone 161b(161a) is configured to receive the heart rate signal through the communication circuit 165b(165a) in the paired smartphone 161b(161a). The processor 162b(162a) is configured to control the vibration source 157 of the vibration generating unit 156b(156a) based on the heart rate signal. The user P, S of the smartphone 161a, 161b is allowed to feel the heart rate (heartbeat) of the other through the heartbeat reproduction apparatus 151a.

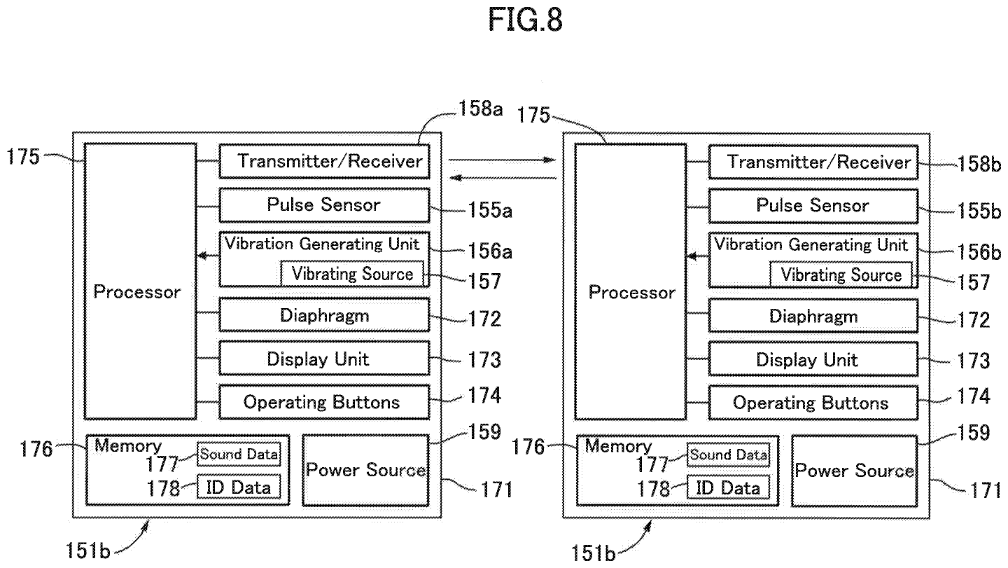

FIG. 8 schematically illustrates the structure of a heartbeat reproduction apparatus 151b according to a sixth embodiment. A heartbeat reproduction unit 171 of the heartbeat reproduction apparatus 151b comprises, in addition to the aforementioned structure, a diaphragm 172, a display unit 173, operating buttons 174, a processor 175 and a memory 176. The processor 175 is connected to the diaphragm 172. The diaphragm 712 is configured to reproduce sound based on an audio signal. The diaphragm 172 may form a loudspeaker. The audio signal may be designed to specify the sound of heartbeat (heart sound), for example. The audio signal is supplied from the processor 175 to the diaphragm 172.

The processor 175 is connected to the display unit 173 and the operating buttons 174. The processor 175 is configured to supply image signals to the display unit 173. The image of an input box or boxes and/or options is displayed on the screen of the display unit 173 based on the image signal. The operating buttons 174 are operated to input numerals, characters, choices, and the like. The processor 175 is in this manner allowed to gain the numerals, characters, choices, and the like. The processor 175 is configured to relate the operation of the operating buttons 174 to the image on the screen of the display unit 173.

The memory 176 is connected to the processor 175. The memory 176 is configured to store a sound data 177 and an ID data 178. The sound data 177 is designed to specify an audio signal. The processor 175 is configured to supply the sound signal to the diaphragm 172 based on the sound data 177. The sound data 177 functions as a heartbeat data specifying the heartbeat. The ID data 178 is designed to specify identifiers assigned to the individual heart reproduction apparatus 151b. The processor 175 is configured to identify the individual heartbeat reproduction apparatus 151b based on the ID data 178.

The processor 175 is configured to add an ID to the heart rate signal based on the ID data 178 registered in the identical heartbeat reproduction apparatus 151b. The heart rate signal is distinguished for the individual heart reproduction apparatuses 151b. The user is directed to register the ID of the paired heart reproduction apparatus 151b to the memory 176 of the identical heart reproduction apparatus 151b. The text box may be displayed on the screen of the display unit 173 for the registration of the ID, the user P, S manipulate the operating buttons 174 to input the ID in the text box for the processor 173. The processor 175 is configured to select the heart rate signal related to the registered ID among the heart rate signals. The selected heart rate signal is supplied to the vibration generating unit 156a, 156b for the reproduction of the heart rate (heartbeat). Accordingly, even if the heartbeat reproduction apparatuses 151b other than the paired heartbeat reproduction apparatus 151b exist within the range of the radio communication of the identical heartbeat reproduction apparatus 151b, the heartbeat reproduction apparatus 151b is allowed to reproduce the heart rate (heartbeat) based on the heart rate signal received from the paired heartbeat reproduction apparatus 151b. In the case where two or more of the IDs are registered beforehand, the ID list may be displayed on the screen of the display unit 173 for presenting available IDs upon the reproduction of the heart rate, and the user may select the ID assigned to the designated heartbeat reproduction apparatus 151b based on the manipulation of the operating buttons 174.

The heartbeat reproduction apparatus 151b may reproduce the heart rate based on the heart sound, in addition to (or in place of) the vibration of the heartbeat. In this case, the processor 175 is configured to generate the audio signal from the audio data based on the heart rate signal. The audio signal is supplied to the diaphragm 172. The diaphragm 172 is configured to reproduce the heart sound. The processor 175 functions as a sound source configured to generate the heart sound in accordance with the heart rate signal. The user P, S of the heartbeat reproduction apparatus 151b is in this manner allowed to feel the heart rate through the sense of hearing. The user P, S may enjoy the reproduction of the heart rate (heartbeat) in a facilitated manner. The user P, S becomes conscious about the heart rate (heartbeat). The heart signal may be displayed as waveforms on the screen of the display unit 173.

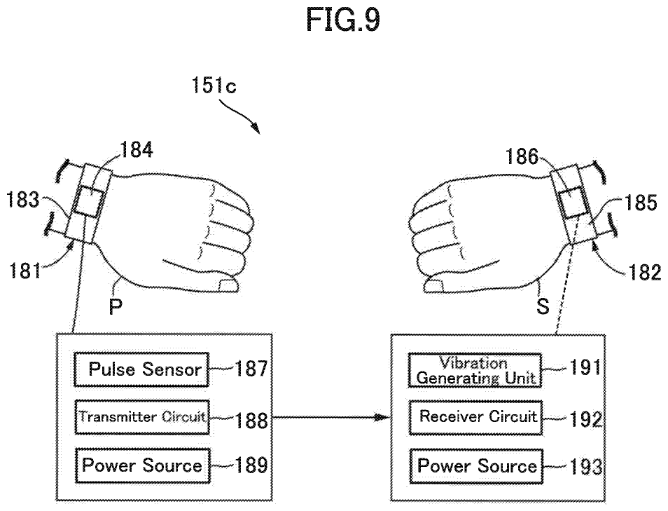

FIG. 9 schematically illustrates the structure of a heartbeat reproduction apparatus 151c according to a seventh embodiment. The heartbeat reproduction apparatus 151c comprises a transmitter unit 181 and a receiver unit 182. The transmitter unit 181 and the receiver unit 182 respectively take the form of a wrist band. Specifically, the transmitter unit 181 comprises a band body 183 and a transmitting module 184 incorporated in the band body 183. The receiver unit 182 comprises a band body 185 and a receiving module 186 incorporated in the band body 185. The band body 183, 185 may have the structure identical to that of the aforementioned band body 152.

The transmitting module 184 includes a pulse sensor 187 and a transmitter circuit 188. The pulse sensor 187 may have the structure identical to that of the aforementioned pulse sensor 155a, 155b. The transmitter circuit 188 is connected to the pulse sensor 187 and configured to receive the heart rate signal from the pulse sensor 187. The transmitter circuit 188 transmits the heart rate signal to the receiving module 186. The pulse sensor 187 and the transmitter circuit 188 may operate in response to the supply of the electricity from a power source 189. The power source 189 may have the structure identical to that of the power source 159. The transmitter unit 181 may take the form of an arm band, a ring, a sticker adhered to the skin of a user, or the like.

The receiving module 186 includes a vibration generating unit 191 and a receiver circuit 192. The vibration generating unit 191 may have the structure identical to that of the aforementioned vibration generating unit 156a, 156b. The receiver circuit 192 is configured to receive the heart rate signal transmitted from the transmitter circuit 188 of the transmitting module 184. The receiver circuit 192 is connected to the vibration generating unit 191 so as to supply the heart rate signal to the vibration generating unit 191. The vibration generating unit 191 and the receiver circuit 192 may operate in response to the supply of the electricity from a power source 193. The power source 193 may have the structure identical to that of the power source 159. The receiver unit 182 may take the form of an arm band, a ring, a sticker adhered to the skin of a user, or the like. The receiving module 186 may be incorporated in a watch, an earphone, glasses, a belt, or other accessory, and the like.

The pulse sensor 187 serves to detect the pulse of the user P. The transmitter circuit 188 is configured to transmit the heart rate signal specifying the heart rate of the user P to the receiver circuit 192. The receiver circuit 192 then supplies the heart rate signal to the vibration generating unit 191. The vibration generating unit 191 is configured to generate vibration (or impact) at a predetermined interval in accordance with the heart rate signal. The user S is thus allowed to feel the heart rate of the user P.

All examples and conditional language recited herein are intended for pedagogical purposes to aid the reader in understanding the invention and the concept contributed by the inventor to furthering the art, and are to be construed as being without limitation to such specifically recited examples and conditions, nor does the organization of such examples in the specification relate to a showing of the superiority and inferiority of the invention. Although the embodiments of the present inventions have been described in detail, it should be understood that the various changes, substitutions, and alterations could be made hereto without departing from the spirit and scope of the invention.

What is claimed is:

1. A heart sound reproduction apparatus comprising:
a receiving section configured to receive a heart rate signal specifying a pace of a heart rate;
a storage section configured to hold a heart sound data specifying a sound wave of a heart sound of one heartbeat for various paces of the heart rate; and
a reproduction section configured to reproduce the heart sound based on the heart sound data in accordance with the pace specified in the heart rate signal.

2. A method of reproducing a heart sound, comprising:
receiving a heart rate signal specifying a pace of a heart rate;
gaining from a storage section a heart sound data specifying a sound wave of a heart sound of one heartbeat for various paces of the heart rate; and
reproducing the heart sound based on the heart sound data in accordance with the pace specified in the heart rate signal.

3. A heartbeat reproduction apparatus comprising:
a first sensor configured to generate a heart rate signal specifying a pace of a heart rate of a first person;
a transmission section configured to transmit the heart rate signal;
a receiving section configured to receive a heart rate signal generated in a second sensor paired with the first sensor, the heart rate signal of the second sensor specifying a pace of a heart rate of a second person;
a storage section configured to hold a heart sound data specifying a sound wave of a heart sound; and
a sound source configured to reproduce a heart sound based on the heart sound data in accordance with the pace specified in the heart rate signal.
